(19) United States
(12) Patent Application Publication
(10) Pub. No.: US 2025/0152501 A1
(43) Pub. Date: May 15, 2025

(54) DISSOLVING MICRONEEDLE FOR TRANSDERMAL DELIVERY OF BIOPHARMACEUTICAL, AND MANUFACTURING METHOD THEREFOR

(71) Applicant: Dongguk University Industry-Academic Cooperation Foundation, Seoul (KR)

(72) Inventors: Hyo Kyung HAN, Seoul (KR); Jae Guen SONG, Seoul (KR)

(73) Assignee: Dongguk University Industry-Academic Cooperation Foundation, Seoul (KR)

(21) Appl. No.: 18/833,780

(22) PCT Filed: Aug. 18, 2022
(86) PCT No.: PCT/KR2022/012308

§ 371 Date: Jul. 26, 2024

(30) Foreign Application Priority Data
Jan. 28, 2022 (KR) 10-2022-0012815

(51) Int. Cl. A61K 9/00; A61K 38/26; A61K 47/30; A61K 47/32

(52) U.S. Cl. CPC A61K 9/0021; A61K 38/26; A61K 47/30; A61K 47/32

(57) ABSTRACT

The present disclosure relates to a dissolving microneedle for transdermal delivery of biopharmaceuticals and a method of manufacturing the same. In the dissolving microneedle of the present disclosure, since biopharmaceuticals form a nanocomposite with aminoclay, the stability of the biopharmaceuticals and the mechanical strength of the microneedle are high, and the skin permeability of the biopharmaceutical is increased, thereby greatly improving transdermal delivery efficiency.

### TECHNICAL FIELD

The present disclosure relates to a dissolving microneedle for transdermal delivery of biopharmaceuticals and a method of manufacturing the same.

### BACKGROUND ART

Biopharmaceuticals have various pharmacological functions and therapeutic effects and are actively applied to treat chronic diseases such as diabetes, rheumatoid arthritis, osteoporosis, or cancer. However, biopharmaceuticals have low bioavailability due to low membrane permeability and stability, and thus, most biopharmaceuticals currently on the market are available in injectable formulations. Therefore, there is a great demand for the development of a non-injectable biopharmaceutical delivery system to improve patient compliance.

The transdermal delivery system, which is one of the non-invasive administration methods that can replace injections, enables self-medication, high patient compliance, and avoidance of hepatic first-pass metabolism. Therefore, efforts to develop a transdermal delivery system to replace the injectable formulation of biopharmaceuticals have been made in various ways. However, biopharmaceuticals, which are water-soluble polymers, have very low skin permeability. Accordingly, when used in the form of conventional patches of the related art, biopharmaceuticals show low transdermal delivery efficiency. This issue can be addressed by applying, for example, iontophoresis, sonophoresis, or microneedles (MNs). Among these, microneedles are currently attracting great attention as a promising technology for effective transdermal delivery of polymers.

Microneedles, of which lengths are generally 50 μm to 900 μm, can be used to pierce the stratum corneum to create micro channels, which can improve the permeability of polymers while minimizing pain. In addition, microneedles can be manufactured in various types, and dissolving microneedles consisting of biodegradable polymers have the property of releasing the loaded drug while dissolving in vivo. That is, compared to other types of microneedles, dissolving microneedles are free from potential risks arising from the disposal process of used needles because the needles are dissolved and removed from the body, and require low manufacturing costs.

However, the hydrophilic polymers used in the manufacture of dissolving microneedles do not have sufficient mechanical strength to pierce the skin, and the use of additives to increase mechanical strength can affect the release and stability of the loaded drug.

Accordingly, in order to solve this issue, the inventors of the present application combined aminoclay (AC) with a biopharmaceutical to form a nanocomposite and then mixed the same with a hydrophilic polymer to manufacture dissolving microneedles, and found that the mechanical strength of the microneedles is increased, and the stability and skin permeability of the loaded biopharmaceutical are increased to significantly enhance transdermal absorption efficiency, thereby completing the present disclosure.

### DETAILED DESCRIPTION OF THE INVENTION

### Technical Problem

One aspect of the present disclosure is to provide a dissolving microneedle comprising (i) a nanocomposite of a biopharmaceutical and aminoclay and (ii) a hydrophilic polymer.

Another aspect of the present disclosure is to provide a patch comprising the dissolving microneedle.

Another aspect of the present disclosure is to provide a method of manufacturing a dissolving microneedle, which comprises: a (i) mixing a biopharmaceutical and aminoclay to form a biopharmaceutical-aminoclay nanocomposite, (ii) mixing the biopharmaceutical-aminoclay nanocomposite with a hydrophilic polymer, (iii) injecting the mixture produced in step (ii) into a microneedle mold, and (iv) separating a microneedle from the microneedle mold after drying.

### Technical Solution

One aspect of the present disclosure provides a dissolving microneedle comprising (i) a nanocomposite of a biopharmaceutical and aminoclay and (ii) a hydrophilic polymer.

The term “microneedle” as used herein refers to a needle-like structure with a length in micrometers (μm), and has a pointed tip like a needle, allowing the same to penetrate the skin. The microneedle forms a hole in the stratum corneum, which is the outermost layer of the skin, and delivers biopharmaceuticals through the formed hole. Additionally, the microneedle is very short in length and do not affect nerve cells, so that they rarely cause pain.

The term “dissolving microneedle” as used herein refers to a microneedle that dissolves in the skin, and when the dissolving microneedle is applied to the skin, the microneedle dissolves or decomposes, allowing biopharmaceuticals to be stably delivered to the skin.

The term “biopharmaceutical” as used herein refers to a medicine manufactured using cells, proteins, genes, etc. derived from humans or other living organisms as raw materials or materials.

The biopharmaceutical may be at least one selected from the group consisting of biological preparations, genetic recombinant medicines, cell culture medicines, peptide medicines, protein medicines, and antibody medicines.

The term “biological preparations” as used herein refers to a medicine comprising a material derived from a living organism or a material produced using a living organism, and the potency and safety thereof cannot be evaluated through physical and chemical tests alone. The biological products may include vaccines, plasma fractionates, and blood preparations, or toxins/antitoxins. Unlike medicines for therapeutic purposes, the “vaccine” refers to a medicine administered to an unspecified number of people, such as healthy infants and young children or the public, to prevent infectious diseases. The “plasma fractionates” refers to a medicine that is obtained through a series of manufacturing processes using plasma as a raw material. The “blood preparations” are medicines manufactured using blood as a raw material, and may be whole blood, concentrated red blood cells, fresh frozen plasma, concentrated platelets, or blood-related medicines. The term “toxins” in the “toxins/antitoxins” refers to a toxic material produced by cells or living organisms, and the term “antitoxin” refers to an antibody that neutralizes toxicity produced by living organisms.

The term “genetically recombinant medicine” as used herein refers to a medicine manufactured using genetic engineering technology, and the term “cell culture medicines” refers to a medicine manufactured using cell culture technology.

The term “peptide” or “protein” as used herein refers to a compound or polymer in which L-amino acids or derivatives or analogs thereof, or D-amino acids or derivatives or analogs thereof are linked to each other through peptide bonds. In addition, the term “peptide medicine” or “protein medicine” as used herein refers to a peptide or protein that can alleviate or cure some of or all a biological disease or prevent the worsening of a disease, through a chemical or biochemical reaction when administered in vivo.

The peptide medicine may be a peptide biopharmaceutical consisting of 3 to 50 amino acids.

The protein medicines are proteins for medicines produced based on genetic recombination, cell culture, or bio-processing, and may include protein medicines used for disease treatment, etc. through mass production using microorganisms or animal cell systems. For example, the protein medicines may be a linear protein drug or a cyclic protein drug. The protein medicines may also be a modified or derivatized protein drug, such as a fatty acid acylated protein medicine or a fatty discrete acylated protein medicine.

In an embodiment, the protein medicine may be at least one selected from the group consisting of lilaglutide (Lira), teriparatide, insulin, insulin analogs, glucagon-like peptide-1 (GLP-1), GLP-2, semaglutide, exenatide, exendin-4, lixisenatide, taspoglutide, albiglutide, dulaglutide, oxyntomodulin, amylin, somatostatin analogs, goserelin, buserelin, leptin, glatiramer acetate, leuprolide, osteocalcin, human growth hormone (hGH), glycopeptide antibiotics, bortezomib, cosyntropin, menotropins, gonadotropin releasing hormone (GnRH), somatropin, calcitonin, oxytocin, lepirudin, carfilzomib, icatibant, and aldesleukin, and is not limited thereto.

The term “antibody” as used herein refers to a material in vivo that is induced by an immune response in response to an antigen, which is an externally derived material that has entered the human body. The antibody includes an immunoglobulin molecule including four polypeptide chains, and two heavy (H) chains and two light (L) chains interconnected by disulfide bonds, as well as multimers thereof (e.g., IgM). Each heavy chain includes a heavy chain variable region and a heavy chain constant region. In addition, the term “antibody medicine” as used herein refers to a medicine using artificial antibodies which are produced through a mass production system using cell lines. In an embodiment, the antibody medicine may be etanercept, trastuzumab, abciximab, rituximab, basiliximab, cetuximab, alemtuzumab, bevacizumab, pavilizumab, adalimumab, certolizumab, eculizumab, catumaxomab, golimumab, efalizumab, lorvotuzumab, brentuximab, glembatumumab, ipilimumab, nivolumab, pembrolizumab, atezolizumab, avelumab, durvalumab, cemiplimab, or infliximab, and is not limited thereto.

The term “aminoclay” as used herein refers to a metal phyllosilicate into which a 3-aminopropyl group is introduced, and the aminoclay is well dispersed in water and may have a positive charge. The metal may be magnesium (Mg), calcium (Ca), iron (Fe), aluminum (AI), manganese (Mn), or zinc (Zn), and is not limited thereto. The aminoclay may be a cationic nanosheet and may interact electrostatically with anionic molecules. The aminoclay may be synthesized through a sol-gel reaction by adding 3-aminopropyltriethoxysilane to an ethanol solution including a cationic metal. The cationic aminoclay may interact electrostatically with anionic molecules. In addition, the aminoclay may temporarily and reversibly induce the opening of tight junctions to promote the influx of macromolecules into cells, thereby improving cellular uptake of macromolecules. Additionally, the aminoclay may improve the thermal stability of proteins and reduce protein aggregation at high temperatures. Therefore, the aminoclay may act as a matrix that effectively maintains the structural stability of the protein formulation. In addition, the aminoclay may increase the mechanical strength of microneedles.

In an embodiment of the present disclosure, the aminoclay may be 3-aminopropyl functionalized magnesium phyllosilicate, and is not limited thereto, and the magnesium in the layered structure may be replaced by other cations, including calcium, iron, aluminum, manganese, zinc, etc.

Due to the positive charge of aminoclay, biopharmaceuticals with the isoelectric point of 9 or less may easily form nanocomposites with aminoclay. Therefore, in an embodiment of the present disclosure, a biopharmaceutical may have an isoelectric point of 9 or less. For example, liraglutide used in the examples of the present disclosure has an isoelectric point of 4.9, and teriparatide has an isoelectric point of 8.3.

The term “hydrophilicity” as used herein refers to the degree of affinity of a material with water. Hydrophilic materials have a strong affinity for water and tend to dissolve in or mix with water. Accordingly, the “hydrophilic polymer” refers to a polymer material that has a strong affinity for water.

In an embodiment of the present disclosure, the hydrophilic polymer may be at least one selected from the group consisting of hyaluronic acid, poly vinyl alcohol (PVA), poly vinyl pyrrolidone, carboxymethyl cellulose, a vinylpyrrolidone-vinyl acetate copolymer, and a polyglycolic acid.

The amount of the biopharmaceutical may be, based on the total weight of the dissolving microneedle, 1 wt % to 10 wt %, for example, 1 wt % to 9 wt %, 1 wt % to 8 wt %, 1 wt % to 7 wt %, 2 wt % to 9 wt %, 2 wt % to 8 wt %, 2 wt % to 7 wt %, 3 wt % to 7 wt %, or 3 wt % to 6 wt %.

The amount of the aminoclay may be, based on the total weight of the dissolving microneedle, 1 wt % to 10 wt %, for example, 1 wt % to 9 wt %, 1 wt % to 8 wt %, 1 wt % to 7 wt %, 2 wt % to 9 wt %, 2 wt % to 8 wt %, 2 wt % to 7 wt %, 3 wt % to 7 wt %, or 3 wt % to 6 wt %.

The amount of the hydrophilic polymer may be, based on the total weight of the dissolving microneedle, 80 wt % to 98 wt %, for example, 81 wt % to 98 wt %, 82 wt % to 98 wt %, 83 wt % to 98 wt %, 84 wt % to 98 wt %, 85 wt % to 98 wt %. %, 82 wt % to 95 wt %, 82 wt % to 97 wt %, 82 wt % to 96 wt %, 82 wt % to 95 wt %, 83 wt % to 97 wt %, 83 wt % to 96 wt %, 84 wt % to 96 wt %, 85 wt % to 95 wt %, or 85 wt % to 94 wt %.

In an embodiment of the present disclosure, the dissolving microneedle may include, based on the total weight of the dissolving microneedle, 1 wt % to 10 wt % of biopharmaceutical, 1 wt % to 10 wt % of aminoclay, and 80 wt % to 98 wt % of hydrophilic polymer.

The dissolving microneedle may include the biopharmaceutical and aminoclay in a weight ratio of 1:0.3 to 3, for example, 1:0.4 to 3, 1:0.5 to 3, 1:0.6 to 3, 1:0.3 to 2.5, 1:0.4 to 2.5, 1:0.5 to 2.5, or 1:0.5 to 2, and is not limited thereto.

The dissolving microneedle may additionally include a plasticizer, a surfactant, a preservative, etc.

The needle of the microneedle may have a cone shape, a pyramid shape, or a polygonal pyramid shape, and the shape thereof is not limited thereto.

The height of the microneedle needle is not limited to a specific height. However, for the passage through the stratum corneum of the skin, the vertical height of the microneedle needle may be 100 μm to 1,000 μm, for example, 200 μm to 1,000 μm, 300 μm to 1,000 μm, 400 μm to 1,000 μm, 100 μm to 900 μm, 200 μm to 900 μm, 300 μm to 900 μm, 400 μm to 900 μm, 100 μm to 800 μm, 200 μm to 800 μm, 300 μm to 800 μm, or 400 μm to 800 μm. However, the height of the microneedle may be appropriately adjusted by a person skilled in the art in consideration of the purpose of use of the microneedle, the depth of the target skin layer, the type of biopharmaceutical, etc., and is not limited to the height ranges.

The diameter of a cone-shaped microneedle and the length of one side of the bottom of a pyramid-shaped or polygonal pyramid-shaped microneedle may be 100 μm to 1000 μm, for example, 100 μm to 900 μm, 100 μm to 800 μm, 100 μm to 700 μm, 200 μm to 900 μm, 200 μm to 800 μm, 200 μm to 700 μm, 300 μm to 900 μm, 300 μm to 800 μm, or 300 μm to 700 μm, and is not limited thereto.

The microneedles may be arranged at regular intervals.

The microneedles may be manufactured using various methods known in the art.

In the dissolving microneedle according to the present disclosure comprising (i) a nanocomposite of a biopharmaceutical and aminoclay and (ii) a hydrophilic polymer, since the biopharmaceutical and aminoclay form a nanocomposite, the stability of the biopharmaceutical and the mechanical strength of the microneedle are high, and the skin permeability of biopharmaceuticals is improved, which may greatly improve transdermal delivery efficiency.

Another aspect of the present disclosure provides a patch comprising the dissolving microneedles.

The term “patch” as used herein refers to a formulation that is attached to the skin and delivers biopharmaceuticals into the body. The size of the patch is not limited to a specific size and may be appropriately adjusted depending on the amount of biopharmaceuticals to be absorbed into the skin or the attachment site thereof.

The description of the dissolving microneedles is the same as described above.

Another aspect of the present disclosure provides a method of manufacturing dissolving microneedles comprising: (i) mixing a biopharmaceutical and aminoclay to form a biopharmaceutical-aminoclay nanocomposite; (ii) mixing the biopharmaceutical-aminoclay nanocomposite with a hydrophilic polymer; (iii) injecting a mixture produced in step (ii) into a microneedle mold; and (iv) separating a microneedle from the microneedle mold after drying.

Descriptions of the biopharmaceutical, aminoclay, and the hydrophilic polymer are as described above.

Another aspect of the present disclosure provides a method of administering a biopharmaceutical to the skin by attaching the dissolving microneedle on the skin.

The number or time of attaching the dissolving microneedle on the skin depends on factors including the condition and weight of a subject, the type and degree of the disease, the type and dose of the biopharmaceutical, the sensitivity of the subject to the biopharmaceutical, drugs used simultaneously, and other factors known in the medical field. The term “individual” as used herein may refer to a subject in need of treatment for a disease, and more specifically, may refer to mammals such as humans or non-human primates, such as dogs, cats, pigs, horses, and cows.

### Advantageous Effects of Disclosure

In the dissolving microneedle according to one aspect of the present disclosure, the biopharmaceutical and aminoclay form a nanocomposite, so that the stability of the biopharmaceutical and the mechanical strength of the microneedle are high, and the skin permeability of the biopharmaceutical is improved, thereby greatly increasing the transdermal delivery efficiency.

### BRIEF DESCRIPTION OF DRAWINGS

FIG. 1 shows a scanning electron microscope (SEM) image of AC-Lira-PVA-MNs. Specifically, FIG. 1A shows an SEM image of AC-Lira-PVA-MNs viewed at 35×, FIG. 1B shows an SEM image viewed at 60×, FIG. 1C shows an SEM image viewed at 150×, and FIG. 1D shows an SEM image of the side of the microneedle viewed at 150×, confirming that the needle height of the microneedle is approximately 600 μm.

FIG. 2 shows the Fourier transform infrared spectroscopy (FT-IR) spectra of dissolving microneedles. FIG. 2A show the FT-IR spectra of lilaglutide (Lira), aminoclay (AC), AC-Lira, polyvinyl alcohol (PVA), and AC-Lira-PVA-MNs, FIG. 2B show the FT-IR spectra of teriparatide (Teri), aminoclay, AC-Teri, PVA, and AC-Teri-PVA-MNs.

FIG. 3 shows the circular dichroism (CD) spectra of a biopharmaceutical released from dissolving microneedles. FIG. 3A shows the CD spectra of pure liraglutide and liraglutide released from AC-Lira-PVA-MNs, and FIG. 3B shows the CD spectra of pure teriparatide and teriparatide released from AC-Lira-PVA-MNs. am.

FIG. 4 shows images of the pig skin stained with hematoxylin-eosin (H&E) after the pig skin is treated with AC-Lira-PVA-MNs and AC-Lira-PVA-MNs is separated therefrom.

FIG. 5 shows a graph of blood glucose changes in streptozotocin (STZ)-induced type 2 diabetic mice after treatment with liraglutide through microneedles (AC-Lira-PVA-MNs) or subcutaneous injection in STZ-induced type 2 diabetic mice.

### MODE FOR INVENTION

The present disclosure will be described in more detail through Examples below. However, these examples are intended to illustrate one or more embodiments and the scope of the present disclosure is not limited to these examples.

### Example 1. Material Preparation and Analysis Methods

### (1) Research Samples

Liraglutide was purchased from Chengdu Shennuo Biopharm Co., Ltd (Dayi County, China). Polyvinyl alcohol (MW=13,000 to 23,000), streptozotocin (STZ), and APTES (99%) were purchased from Sigma-Aldrich Co. (St Louis, MO, USA). Paraformaldehyde was purchased from Merck KGaA (Darmstadt, Germany). Magnesium chloride hexahydrate (98%) and other inorganic salts were purchased from Junsei Chemical Co., Ltd (Tokyo, Japan). For all chemicals and reagents, HPLC-grade was used.

### (2) Production of Aminoclay

To prepare aminoclay, 3-aminopropyl-functionalized magnesium phyllosilicate, which is a type of aminoclay, was synthesized. Specifically, 1.68 g of magnesium chloride was dissolved in 40 g of ethanol, and 2.6 ml of APTES was added dropwise thereto while vigorous stirring at 250 rpm to rapidly form a white precipitate. After stirring for 24 hours, the resulting precipitate was centrifuged and washed three times with ethanol. The resultant product was dried at a temperature of 40° C. The obtained powder was dispersed in water to exfoliate the aminoclay and treated with ultrasonic waves for 10 minutes.

### (3) STZ-Induced Type 2 Diabetes Model Mice

To use STZ-induced type 2 diabetes model mice in experiments, male Sprague-Dawley (230 to 250 g) were purchased from Orient Bio Inc. (Seongnam, Korea).

First, in order to induce the mice into a type 2 diabetes model, the mice were provided with a high fat diet and water for 3 weeks. After 3 weeks, the mice were fasted for 6 hours to 8 hours before STZ treatment. Then, the mice were intraperitoneally treated with 40 mg/kg of STZ in 50 mM citrate buffer solution (pH 4.5) to obtain STZ-induced type 2 diabetes model mice.

To confirm that the type 2 diabetes model was appropriately induced, blood glucose was measured using a Roche blood glucose meter (ACCU-CHEK guide) 10 days after the intraperitoneal treatment. As a result, the measured blood glucose levels of the mice were 300 mg/dl or more. Therefore, it was confirmed that the type 2 diabetes model mice were appropriately induced.

### (4) HPLC Analysis

The concentration of biopharmaceuticals was quantified using an HPLC system (Perkin Elmer, MA, USA). Chromatographic separation was performed at 40° C. using a column (Gemini C18, 4.6×150 mm, 5 μm; Phenomenex, Torrance, CA, USA). Mobile phase A was set to contain acetonitrile comprising 0.1% trifluoroacetic acid, and mobile phase B was set to contain water comprising 0.1% trifluoroacetic acid. Then, the flow rate was set to 0.6 mL/min, and the gradient elution was performed using the following conditions: gradient elution with 60% to 50% solvent B for 0 minutes to 2.5 minutes, gradient elution with 50% to 40% solvent B for 2.5 minutes to 3.0 minutes, gradient elution with 40% to 50% solvent B for 4.0 minutes to 6.0 minutes, gradient elution with 50% to 60% solvent B for 6.0 minutes to 7.0 minutes, and gradient elution with 60% solvent B for 7.0 minutes to 10.0 minutes.

Tolbutamide was used as an internal standard, and the detection wavelength was set at 220 nm. As a result, a linear calibration curve was obtained in the range of 1 μg/mL to 200 μg/m L (R²>0.999).

### Example 2. Preparation of Dissolving Microneedles Comprising Biopharmaceutical-Aminoclay Nanocomposite and Hydrophilic Polymer

### (1) Preparation of Dissolving Microneedles (AC-Lira-PVA-MNs) Comprising Aminoclay-Liraglutide-Polyvinyl Alcohol Mixture

Dissolving microneedles were manufactured step by step using a polydimethylsiloxane (PDMS) MN mold, using a two-step molding method (step by step method). Specifically, 10 mg/ml liraglutide solution and 10 mg/ml aminoclay (a protein ratio of 1:1) were mixed while stirring to obtain an aminoclay-liraglutide (AC-Lira) composite. The average size of the AC-Lira composite was confirmed to be 135±9.04 nm.

Next, to 100 mg/ml of polyvinyl alcohol solution, the AC-Lira composite was added dropwise in the same volume while stirring, thereby obtaining a mixture of AC-Lira composite and polyvinyl alcohol (AC-Lira-PVA). Then, the AC-Lira-PVA was added to the MN mold, and a needle tip was filled using a vacuum chamber (0.8 bar) for 1 minute. Afterwards, the AC-PVA solution without biopharmaceuticals was slowly added to the mold to form a support layer. Next, the microneedle array was dried at room temperature for 24 hours to prepare dissolving microneedles (AC-Lira-PVA-MNs) comprising an aminoclay-liraglutide-polyvinyl alcohol mixture.

### (2) Preparation of Dissolving Microneedles (AC-Teri-PVA-MNs) Comprising Aminoclay-Teriparatide-Polyvinyl Alcohol Mixture

Dissolving microneedles were manufactured step by step using a PDMS MN mold according to a two-step molding method. Specifically, 10 mg/ml teriparatide solution and 10 mg/ml aminoclay were mixed with stirring at a protein ratio of 1:1 to prepare aminoclay-teriparatide (AC-Teri) composite. Next, to 100 mg/ml of polyvinyl alcohol solution, the AC-Teri composite was added dropwise in the same volume while stirring, thereby obtaining a mixture of AC-Teri composite and polyvinyl alcohol (AC-Teri-PVA). Then, the AC-Teri-PVA was added to the MN mold, and needle tips were filled using a vacuum chamber (0.8 bar) for 1 minute. Afterwards, the AC-PVA solution without biopharmaceuticals was slowly added to the mold to form a support layer. Next, the microneedle array was dried at room temperature for 24 hours to prepare dissolving microneedles (AC-Teri-PVA-MNs) including an aminoclay-teriparatide-polyvinyl alcohol mixture.

### Experimental Example 1. Morphological Analysis of Dissolving Microneedles

The shape and structural characteristics of the dissolving microneedles (AC-Lira-PVA-MNs) prepared in Example 2(1) were analyzed.

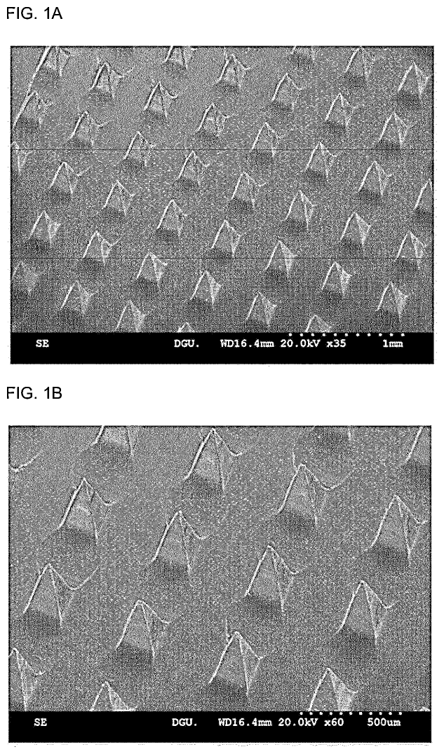

The morphological characteristics of the dissolving microneedles were viewed using a scanning electron microscope (SEM) (Hitach S-3000N; Hitachi, Japan). Results are shown in FIG. 1.

FIG. 1 shows a scanning electron microscope (SEM) image of AC-Lira-PVA-MNs. FIG. 1A shows an SEM image of AC-Lira-PVA-MNs viewed at 35×, FIG. 1B shows an SEM image viewed at 60×, FIG. 1C shows an SEM image viewed at 150×, and FIG. 1D shows an SEM image of the side of the microneedle viewed at 150×, confirming that the needle height of the microneedle is approximately 600 μm.

From the experimental results, it was confirmed that the needle of AC-Lira-PVA-MNs had a pyramid shape with a tip length of about 600 μm and a base length of about 500 μm.

### Experimental Example 2. Measurement of Strength of Dissolving Microneedles

To measure the mechanical strength of AC-Lira-PVA-MNs and AC-Teri-PVA-MNs prepared in Example 2, a low-force mechanical testing system was used (TA-XT express texture analyzer, Stable Micro systems UK). The gap between the MN tip and the stainless-steel probe was set to be 2.0 mm, and the trigger force was set to be 0.049 N. The moving speed of the top stainless-steel probe was set at 0.05 mm/s. The mechanical strength of AC-Lira-PVA-MNs and AC-Teri-PVA-MNs was compared with dissolving microneedles (Lira-PVA-MNs) that do not contain aminoclay. Results thereof are shown in Table 1.

TABLE 1

| Dissolving microneedles | Mechanical strength (N/needles) |
|---|---|
| Lira-PVA-MNs | 0.14 ± 0.035 |
| AC-Lira-PVA-MNs | 0.83 ± 0.040 |
| AC-Teri-PVA-MNs | 0.89 ± 0.025 |

As shown in Table 1, the mechanical strength of Lira-PVA-MNs without aminoclay is 0.14±0.035 N/needles, and the mechanical strength of AC-Lira-PVA-MNs with aminoclay is 0.83±0.040 N/needles, 5 times or more compared to that of Lira-PVA-MNs. The mechanical strength of AC-Teri-PVA-MNs including aminoclay was also 0.89±0.025 N/needles, confirming that the mechanical strength was high due to the inclusion of aminoclay.

In addition, the strength of AC-Lira-PVA-MNs was measured by varying the amount ratio of the aminoclay. As a result, it was confirmed that the strength was increased as the amount of aminoclay was increased.

### Experimental Example 3. Fourier Transform Infrared Spectroscopy (FT-IR) Analysis of Dissolving Microneedles

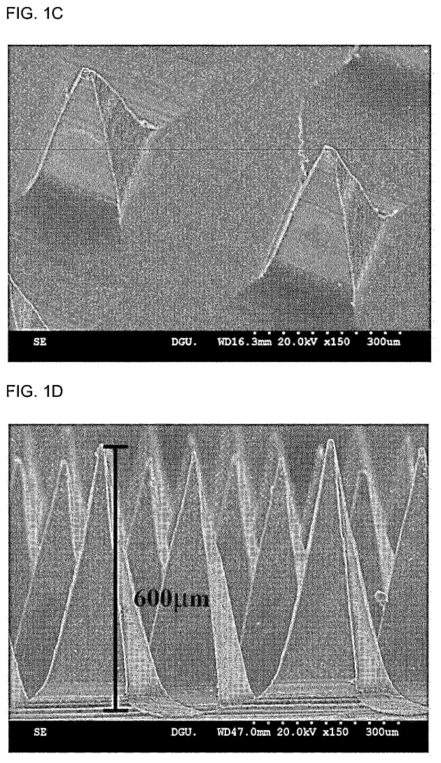

Using FT-IR, the structural properties of AC-Lira-PVA-MNs and AC-Teri-PVA-MNs prepared in Example 2 were confirmed. Results thereof are shown in FIG. 2.

As shown in FIG. 2A, the FT-IR spectrum of AC-Lira has the Si—C band at 1130 cm⁻¹, the Si—O—Si band at 1008 cm⁻¹, and the featured peak of the α-helical structure of Liraglutid at 1652 cm⁻¹ to 1654 cm⁻¹, and the Mg—O—Si band at 559 cm⁻¹ to 497 cm⁻¹. The FT-IR spectrum of AC-Lira-PVA-MNs showed the featured peaks of both AC-Lira and PVA.

Likewise, as shown in FIG. 2B, the FT-IR spectrum of AC-Teri showed the absorbance bands of both aminoclay and Teri, and the FT-IR spectrum of AC-Teri-PVA-MNs also showed the featured peaks of AC-Teri and PVA.

Thus, it was found that PVA-MNs were loaded with biopharmaceuticals combined with aminoclay, forming AC-Lira-PVA-MNs and AC-Teri-PVA-MNs.

### Experimental Example 4. Measurement of Stability of Biopharmaceuticals Released from Dissolving Microneedles

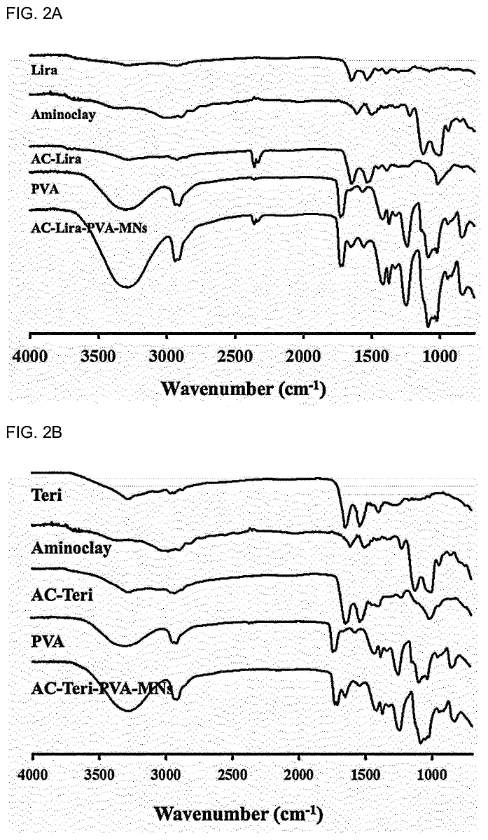

To measure the structural stability of liraglutide released from AC-Lira-PVA-MNs prepared in Example 2 (1), the structural stability of liraglutide released from AC-Lira-PVA-MNs in a phosphate buffer solution at pH 7.4 was investigated using circular dichroism (CD) analysis and compared with pure liraglutide (native Lira). The structural stability of teriparatide released from AC-Teri-PVA-MNs prepared in Example 2(2) was measured in the same manner. Results are shown in FIG. 3.

As shown in FIG. 3A, pure liraglutide showed the featured peak of a typical α-helical structure at 208 nm and 222 nm. The CD spectrum of liraglutide released from AC-Lira-PVA-MNs was almost identical to that of pure liraglutide. As a result, it was found that the secondary structure of liraglutide was well maintained in AC-Lira-PVA-MNs.

Similarly, as shown in FIG. 3B, the CD spectrum of teriparatide released from AC-Teri-PVA-MNs was confirmed to be almost identical to that of pure teriparatide. As a result, it was found that the secondary structure of teriparatide was well maintained in AC-Teri-PVA-MNs.

The results indicate that aminoclay is a support matrix that can effectively maintain the intrinsic structure of a protein.

### Experimental Example 5. In Vitro Skin Permeation Experiment

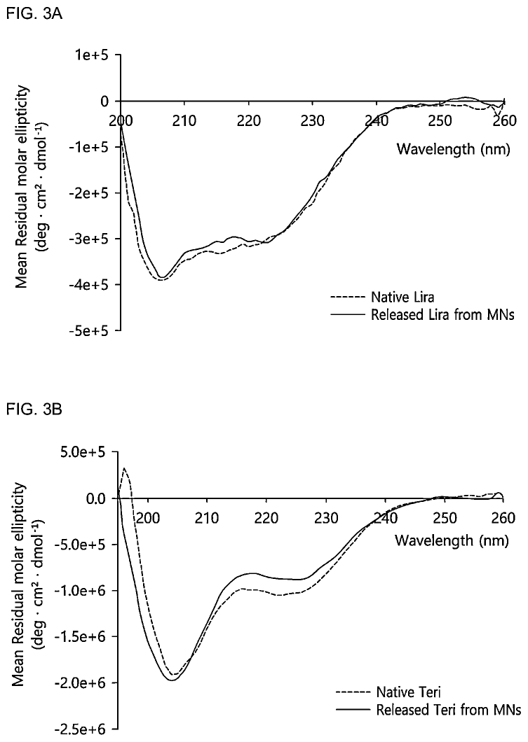

To measure skin penetration of AC-Lira-PVA-MNs prepared in Example 2(1), the pig cadaver skin was used. AC-Lira-PVA-MNs were attached on the skin by pressing with a spring applicator (Micropoint Technologies, Singapore) and removed after 1 minute. AC-Lira-PVA-MNs were separated from the skin, and histological analysis of the skin was performed using hematoxylin-Eosin (H&E) staining. Specifically, the skin sample was fixed in PBS including 4% paraformaldehyde, dehydrated, and then embedded in paraffin. Embedded skin samples were cut into a section having the thickness of 10 μm using a microtome (Leica, Wetzlar, Germany), stained with H&E, and scanned using an Eclipse Ti-U inverted microscope (Nikon, Tokyo, Japan). Results are shown in FIG. 4.

As shown in FIG. 4, AC-Lira-PVA-MNs were stably inserted into the skin, and the depth of the micropores created by AC-Lira-PVA-MNs was confirmed to be approximately 600 μm. Considering the depth of the stratum corneum (10 μm to 15 μm), the depth of the epidermis (50 μm to 100 μm), and the depth of the papillary dermis (100 μm to 200 μm), it is indicated that AC-Lira-PVA-MNs can facilitate the entry of biopharmaceuticals into the systemic circulation. Therefore, it was confirmed that AC-Lira-PVA-MNs formed appropriate micropores in the skin to facilitate biopharmaceutical penetration.

### Experimental Example 6. In Vivo Efficacy Study

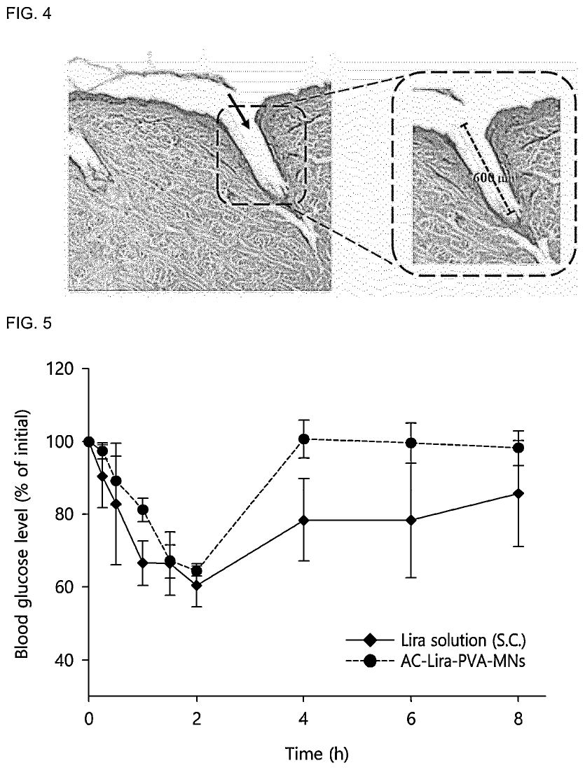

To measure the in vivo efficacy of liraglutide of AC-Lira-PVA-MNs prepared in Example 2 (1), type 2 diabetes model mice induced by STZ according to Example 1(3) were randomly divided into two groups (Group 1 and Group 2). Group 1 received 100 μg/kg of liraglutide solution by subcutaneous injection, and Group 2 received 100 μg/kg of liraglutide by AC-Lira-PVA-MN. Then, blood samples from Group 1 and Group 2 were collected, blood glucose levels were measured using a blood glucose meter (ACCU-CHEK Guide), and changes in blood glucose levels were compared. Results are shown in FIG. 5. The blood glucose level was expressed as a percentage of the initial blood glucose level.

As a result, it was confirmed that the blood glucose concentration of Group 1, which received the liraglutide solution by subcutaneous injection, was rapidly decreased to 61% of the initial blood glucose level within 2 hours after administration, and then gradually recovered to the initial blood glucose level over 8 hours. It was confirmed that the blood glucose concentration of Group 2 treated with AC-Lira-PVA-MNs rapidly decreased to 64% of the initial blood glucose level within 2 hours after treatment, and then gradually recovered to the initial blood glucose level over 4 hours. As a result, it was found that even when treated with AC-Lira-PVA-MNs, an effective hypoglycemic effect occurred, similar to when the liraglutide solution was administered by subcutaneous injection. This means that transdermal delivery of biopharmaceuticals may be effectively achieved through aminoclay-based dissolving microneedles in which biopharmaceuticals were loaded.

As shown in FIG. 5, the treatment of liraglutide with AC-Lira-PVA-MNs produced similar hypoglycemic effects to that of the same dose of liraglutide administered by subcutaneous injection. This means that AC-Lira-PVA-MNs may effectively deliver liraglutide into the body.

1. A dissolving microneedle, comprising:
a nanocomposite of a biopharmaceutical and aminoclay; and
a hydrophilic polymer.

2. The dissolving microneedle of claim 1, wherein the biopharmaceutical is at least one selected from the group consisting of biological preparations, genetic recombinant medicines, cell culture medicines, peptide medicines, protein medicines, and antibody medicines.

3. The dissolving microneedle of claim 1, wherein the biopharmaceutical has an isoelectric point of 9 or less.

4. The dissolving microneedle of claim 1, wherein the biopharmaceutical is a protein medicine, and
the protein medicine is at least one selected from the group consisting of lilaglutide, teriparatide, insulin, insulin analogs, glucagon-like peptide-1 (GLP-1), GLP-2, semaglutide, exenatide, exendin-4, lixisenatide, taspoglutide, albiglutide, dulaglutide, oxyntomodulin, amylin, somatostatin analogs, goserelin, buserelin, leptin, glatiramer acetate, leuprolide, osteocalcin, human growth hormone (hGH), glycopeptide antibiotics, bortezomib, cosyntropin, menotropins, gonadotropin releasing hormone (GnRH), somatropin, calcitonin, oxytocin, lepirudin, carfilzomib, icatibant, and aldesleukin.

5. The dissolving microneedle of claim 1, wherein the aminoclay is a 3-aminopropyl group-introduced metal phyllosilicate.

6. The dissolving microneedle of claim 5, wherein the metal is magnesium (Mg).

7. The dissolving microneedle of claim 1, wherein the hydrophilic polymer comprises at least one selected from the group consisting of hyaluronic acid, poly vinyl alcohol, poly vinyl pyrrolidone, carboxymethyl cellulose, a vinylpyrrolidone-vinyl acetate copolymer, and polyglycolic acid.

8. The dissolving microneedle of claim 1, wherein the dissolving microneedle comprises 1 wt % to 10 wt % of the biopharmaceutical, 1 wt % to 10 wt % of aminoclay, and 80 wt % to 98 wt % of the hydrophilic polymer, based on the total weight of the dissolving microneedle.

9. A patch comprising the dissolving microneedle of claim 1.

10. A method of manufacturing a dissolving microneedle, the method comprising:
(i) mixing a biopharmaceutical and aminoclay to form a biopharmaceutical-aminoclay nanocomposite;
(ii) mixing the biopharmaceutical-aminoclay nanocomposite with a hydrophilic polymer;
(iii) injecting a mixture produced in step (ii) into a microneedle mold; and
(iv) separating a microneedle from the microneedle mold after drying.

11. The method of claim 10, wherein the biopharmaceutical is at least one selected from the group consisting of biological preparations, genetic recombinant medicines, cell culture medicines, peptide medicines, protein medicines, and antibody medicines.

12. The method of claim 10, wherein the aminoclay is 3-aminopropyl group-introduced metal phyllosilicate.

13. The dissolving microneedle of claim 2, wherein the biopharmaceutical is a protein medicine, and
the protein medicine is at least one selected from the group consisting of lilaglutide, teriparatide, insulin, insulin analogs, glucagon-like peptide-1 (GLP-1), GLP-2, semaglutide, exenatide, exendin-4, lixisenatide, taspoglutide, albiglutide, dulaglutide, oxyntomodulin, amylin, somatostatin analogs, goserelin, buserelin, leptin, glatiramer acetate, leuprolide, osteocalcin, human growth hormone (hGH), glycopeptide antibiotics, bortezomib, cosyntropin, menotropins, gonadotropin releasing hormone (GnRH), somatropin, calcitonin, oxytocin, lepirudin, carfilzomib, icatibant, and aldesleukin.

14. The dissolving microneedle of claim 3, wherein the biopharmaceutical is a protein medicine, and
the protein medicine is at least one selected from the group consisting of lilaglutide, teriparatide, insulin, insulin analogs, glucagon-like peptide-1 (GLP-1), GLP-2, semaglutide, exenatide, exendin-4, lixisenatide, taspoglutide, albiglutide, dulaglutide, oxyntomodulin, amylin, somatostatin analogs, goserelin, buserelin, leptin, glatiramer acetate, leuprolide, osteocalcin, human growth hormone (hGH), glycopeptide antibiotics, bortezomib, cosyntropin, menotropins, gonadotropin releasing hormone (GnRH), somatropin, calcitonin, oxytocin, lepirudin, carfilzomib, icatibant, and aldesleukin.
